(19) United States
(12) Patent Application Publication
(10) Pub. No.: US 2011/0319360 A1
(43) Pub. Date: Dec. 29, 2011

(54) Method of including apoptosis/Cell death in leukemia cell using a purine nucleoside analogue

(75) Inventors: J. Brice Weinberg, Durham, NC (US); Daphne R. Friedman, Durham, NC (US); George J. Cianciolo, Chapel Hill, NC (US); David A. Rizzieri, Chapel Hill, NC (US)

(73) Assignee: U.S. Department of Veterans Affairs, Washington DC, DC (US)

(21) Appl. No.: 12/801,730

(22) Filed: Jun. 23, 2010

(51) Int. Cl. A61K 31/69; C12N 5/02; A61P 35/02

(52) U.S. Cl. 514/64; 435/375

(57) ABSTRACT

A method of inducing apoptosis or cell death in a leukemia cell includes subjecting the cell to a purine nucleotide analogue, LMP-420 (2-amino-6-chloro-9-[5(dihydroxyboryl)-pentyl] purine).

### STATEMENT REGARDING FEDERALLY SPONSORED RESEARCH OR DEVELOPMENT

The work leading to the present invention was funded in part by the U.S. Government (NIH and the Department of Veterans Affairs), and the Leukemia/Lymphoma Society. The U.S. Government therefore has certain rights in the invention.

### FIELD AND BACKGROUND OF THE INVENTION

The present invention is generally directed to the treatment of hematologic malignancies, such as Chronic Lymphocytic Leukemia (CLL) and Non-Hodgkin's Lymphomas (NHL), and more particularly to a method of inducing apoptosis/cell death in leukemia cells using a purine nucleoside analogue.

Chronic lymphocytic leukemia (CLL) is an incurable and common adult B-cell malignancy. While highly effective chemo-immunotherapy regimens exist for the first-line setting, treatment options for relapsed or refractory CLL are limited. Additionally, therapy for CLL is usually accompanied by treatment-induced immuno- and myelosuppression (References 1 and 2). Purine analogue-based treatments in particular are associated with long-term T-cell depletion and increased risk of opportunistic infections, limiting the number of treatment cycles able to be administered (References 1-4). Therefore, identifying alternate treatment options without accompanying hematologic toxicities has clinical relevance.

CLL cells can produce tumor necrosis factor (TNF), a cytokine that can improve viability and promote proliferation of CLL cells in vitro (References 5 and 6). TNF is likely a clinically relevant target in CLL, given that elevated plasma TNF levels are correlated with poor prognosis (Reference 7). These observations have led to the hypothesis that blocking TNF in CLL patients would be efficacious.

We have assessed the effect of the purine nucleoside analogue LMP-420 (2-amino-6-chloro-9-[5(dihydroxyboryl)-pentyl] purine; shown in FIG. 1) on CLL cells freshly isolated from patients. LMP-420 is a novel anti-inflammatory agent that impairs peripheral blood mononuclear cell (PBMC) production of TNF without impairing PBMC viability (Reference 8). Clinical trials of etanercept, a soluble chimeric TNF receptor that inhibits TNF-cell interactions, have thus far shown no or limited efficacy in CLL (References 9 and 10). However, LMP-420 acts through different mechanisms, by both inhibiting TNF synthesis at the transcriptional level and by altering expression of additional inflammatory mediators and cell surface molecules (Reference 8). Based on the anti-apoptotic and pro-proliferative effects of TNF on CLL cells, we hypothesized that LMP-420 would be cytotoxic to CLL cells and have therapeutic potential for this disease. We also hypothesized that LMP-420 might potentiate the toxicity of fludarabine to CLL cells without increasing toxicity to normal hematopoietic cells.

### ASPECTS OF THE INVENTION

The present disclosure is directed to various aspects of the present invention.

One aspect of the present invention includes a new use of a purine nucleoside analogue, 2-amino-6-chloro-9-[5(dihydroxyboryl)-pentyl], purine, as an anticancer agent.

Another aspect of the present invention includes a purine nucleoside analogue, which has potent cytotoxic and anti-proliferative effects on chronic lymphocytic leukemia cells and minimal or negligible toxicity for normal hematopoietic cells.

Another aspect of the present invention includes a new use of a purine nucleoside analogue, 2-amino-6-chloro-9-[5(dihydroxyboryl)-pentyl] purine, as an agent for inducing apoptosis or cell death in a leukemia cell, particularly a chronic lymphocytic leukemia cell.

Another aspect of the present invention includes a new use of a purine nucleoside analogue, 2-amino-6-chloro-9-[5(dihydroxyboryl)-pentyl] purine, as an agent for enhancing the cytotoxic activity of fludarabine in inducing apoptosis or cell death in a leukemia cell, particularly a chronic lymphocytic leukemia cell.

Another aspect of the present invention includes a new use of a purine nucleoside analogue, 2-amino-6-chloro-9-[5(dihydroxyboryl)-pentyl] purine, as an agent for inhibiting proliferation of leukemia cells, particularly chronic lymphocytic leukemia cells.

Another aspect of the present invention includes a new use of a purine nucleoside analogue, 2-amino-6-chloro-9-[5(dihydroxyboryl)-pentyl] purine, as an agent for treating hematologic malignancies, such as Chronic Lymphocytic Leukemia (CLL) and Non-Hodgkin's Lymphomas (NHC).

Another aspect of the present invention includes a method of inducing apoptosis or cell death in a leukemia cell, which includes subjecting a leukemia cell to a purine nucleoside analogue.

Another aspect of the present invention includes a method of enhancing the cytotoxic activity of fludarabine in inducing apoptosis or cell death in a leukemia cell, which includes subjecting a leukemia cell to a purine nucleoside analogue and fludarabine.

Another aspect of the present invention includes a method of inhibiting proliferation of a leukemia cell, which includes subjecting a leukemia cell to a purine nucleoside analogue.

Another aspect of the present invention includes a method of treating leukemia, which includes administering to a subject in need thereof an effective amount of a purine nucleoside analogue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

One of the above and other aspects, novel features and advantages of the present invention will become apparent from the following detailed description of the preferred embodiment(s) invention, as illustrated in the drawings, in which:

FIG. 1 illustrates chemical structure of LMP-420 (2-amino-6-chloro-9-[5(dihydroxyboryl)-pentyl] purine);

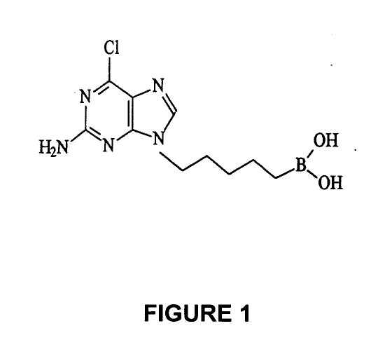

FIGS. 2a-b illustrate cytotoxicity of LMP-420 for CLL cells;

FIGS. 3A-D illustrate LMP-420-mediated apoptosis inhibition in CLL cells;

FIG. 4 illustrates LMP-420-mediates a decrease in CLL cell proliferation;

FIG. 5a-b illustrate potentiation of fludarabine cytotoxicity by LMP-420;

FIG. 6 is a photomicrograph illustrating gene expression profiling of CLL cells treated with LMP-420;

FIGS. 7a-b illustrate apoptotic effects of LMP-420 and fludarabine on normal B- and T-lymphocytes;

FIG. 8 illustrates LMP-420 induces caspase activation in CLL cells;

FIG. 9 illustrates LMP-420 does not potentiate fludarabine toxicity on normal PBMCs; and

FIG. 10 illustrates lack of effect of LMP-420 on the human potassium channel Herg.

### DETAILED DESCRIPTION OF THE PREFERRED EMBODIMENT(S) OF THE INVENTION

B-cell chronic lymphocytic leukemia (CLL) is characterized by accumulation of malignant cells, which are supported in the microenvironment by cell-cell interactions and soluble cytokines such as tumor necrosis factor (TNF). We evaluated the effect of the small molecule TNF inhibitor LMP-420 on primary CLL cells. The mean concentration of LMP-420 required to induce 50% cytotoxicity (ED50) at 72 hours was 245 nM. LMP-420 induced time- and dose-dependent apoptosis, as demonstrated by annexin V staining, caspase activation, and DNA fragmentation. These changes were associated with decreased expression of anti-apoptotic proteins Mcl-1, Bcl-xL, and Bcl-2. CLL cells from patients with poor prognostic indicators exhibited LMP-420 sensitivity equal to that of cells from patients with favorable prognostic indicators. In addition, LMP-420 potentiated the cytotoxic effect of fludarabine for CLL cells and inhibited in vitro proliferation of stimulated CLL cells. Gene expression profiling indicated that the mechanism of action of LMP-420 may involve suppression of NF-kappa B and immune response pathways in CLL cells. LMP-420 had minimal effects on normal peripheral blood mononuclear cell, B- and T-cell function, and normal hematopoietic colony formation. Our data support efficacious and therapeutic uses of LMP-420 for CLL with negligible hematologic toxicities.

### Methods and Materials

### Cells

CLL patients from the Duke University and Durham VA Medical Centers were enrolled in research protocols to collect clinical data and blood. Institutional review boards (IRBs) at both institutions approved these protocols, and patients signed informed consent prior to phlebotomy, in accordance with the Declaration of Helsinki. Clinical data were obtained by chart review.

On the same day that heparin-anticoagulated venous blood was collected from participants, CLL cells were purified by methods described previously (Reference 11). Briefly, CLL cells were isolated from whole blood using the RosetteSep® B-cell enrichment cocktail (Stem Cell Technologies, Vancouver, BC, Canada) together with ficoll-Hypaque density gradient centrifugation. This method yielded CLL cell purity of greater than 95% CD5⁺CD19⁺ B-cells.

Various prognostic markers including IgV_{H} mutation status, CD38 and ZAP70 expression, and interphase cytogenetics were measured as described previously (Reference 11). Poor risk cytogenetic subgroups are defined as 17p13 deletion or 11q22 deletion, and favorable cytogenetic subgroups are defined as those that are normal, 13q14 deletion, or trisomy 12.

For toxicity studies involving normal blood cells, normal volunteers were enrolled in an IRB-approved protocol for blood collection. PBMCs were isolated from heparin-anticoagulated blood by ficoll-Hypaque density gradient centrifugation.

### Drugs

LMP-420 was synthesized to 96-98% purity by Scynexis Inc. (Durham, N.C.) under a Material Transfer Agreement between Duke University and LeukoMed, Inc. (Raleigh, N.C.) and stored as a 25 mM stock solution in 5% sorbitol, pH 8.5-9.0, at 4° C. Chlorambucil, cladribine, bendamustine, and fludarabine phosphate (fludarabine) were purchased from Sigma-Aldrich, St. Louis, Mo. 4-hydroperoxycyclophosphamide (4-HC) was synthesized by Eno Research & Development (Hillsborough, N.C.). Bendamustine and 4-HC were prepared fresh in sterile distilled water for each experiment.

### MTS Assay

2.5×10⁵ CLL cells were incubated in triplicate or quadruplicate in a 96-well tissue culture plate (Costar, Corning, N.Y.) with serial dilutions of LMP-420, fludarabine, and/or recombinant TNF-alpha (R&D Systems, Minneapolis, Minn.) in Hybridoma serum free media (SFM) (GIBCO/Invitrogen, Carlsbad, Calif.) containing 10% heat-inactivated fetal bovine serum (FBS) (Sigma) at 37° C. with 5% CO₂. After 72 hours, 20 μl CellTiter 96® AQueous One Solution Cell Proliferation Assay (Promega, Madison, Wis.) MTS (3-(4,5 dimethylthiazol-2-yl)-5-(3-carboxymethoxyphenyl-2-(4-sulfophenyl)-2H-tetrazolium) reagent was added to each well and the absorbance was read at 490 nM on a Thermomax microplate reader (Molecular Devices, Sunnyvale, Calif.). We calculated fractional cytotoxicity as performed previously (Reference 12), comparing absorbance at 490 nm from treated cells to control cells incubated with media alone.

The concentration effective at killing 50% of CLL cells compared to media alone (ED50) was calculated when 50% fractional cytotoxicity occurred within the range of serial dilutions tested (Reference 12). If the ED50 fell below or above this range, it was set to either the lowest or the highest concentration tested, respectively, for the purposes of statistical analyses and graphical representation.

### Apoptosis Assays

Cells were cultured in either of two different media: Hybridoma SFM with 10% FBS or RPMI 1640 medium (Sigma) supplemented with 5% heat-inactivated human AB serum (Sigma), 50 units/ml penicillin, 50 μg/ml streptomycin, 2 mM L-glutamine, 25 mM HEPES (N2-hydroxyethylpiperazine-N′-2-ethanesulfonic acid), 100 μM MEM non-essential amino acids, and 1 mM sodium pyruvate (GIBCO). CLL cell sensitivity to LMP-420 was not affected by culture media, as determined by direct comparison of the apoptotic effects observed in both media for seven samples. After drug activity in the presence of human AB serum was confirmed, further studies were performed in Hybridoma SFM with 10% FBS.

CLL cells were cultured at 2×10⁶ cells/ml in a volume of 100 μl/well in a U-bottom 96-well tissue culture plate (BD Biosciences, San Jose, Calif.) with either vehicle control or with drug. After 24, 48, or 72 hours of incubation, cells were analyzed for apoptosis by three different methods. For the annexin-based assay, 100 μl of Guava Nexin Reagent (Millipore, Billerica, Mass.) was added for 20 min. Cells were analyzed for annexin V-PE and 7-AAD staining with flow cytometric analysis by the Guava EasyCyte™ Plus system (Millipore). To facilitate comparison of samples with different levels of spontaneous apoptosis, drug-specific apoptosis is reported. Drug-specific apoptosis excludes background spontaneous apoptosis and was calculated as follows: 100%×(drug-induced apoptosis−spontaneous apoptosis)/(100−spontaneous apoptosis) (Reference 13).

For the second method, apoptosis-associated activation of caspases 3 and 7 was measured by Caspase-Glo® 3/7 Assay, according to the manufacturer's protocol (Promega). Luminescence was measured by Thermo Luminoskan Ascent microplate reader (Thermo Scientific, Waltham, Mass.). For the third method, cell lysates and cell culture supernatants were assayed for histone-associated DNA fragments using the Roche Cell Death Detection ELISA^{Plus} kit (Roche Applied Science, Indianapolis, Ind.) according to the manufacturer's protocol. Nucleosome enrichment was calculated as Absorbance₄₀₅ drug-treated/Absorbance₄₀₅ control sample.

### Flow Cytometric Analysis of Cell Surface and Intracellular Antigens

CLL cells were cultured at 2×10⁶ cells/ml in Hybridoma SFM with 10% FBS in 6-well tissue culture plates (BD Biosciences). After 72 hours treatment with vehicle control or drug, cells were harvested and washed twice with 8 ml Hanks Balanced Salt Solution (HBSS). Staining with antibodies was performed at 4° C. For cell surface staining of CD40, FITC-conjugated CD40 antibody (BD Pharmingen 555588) or FITC-conjugated IgG1 kappa isotype control (BD Pharmingen 555748) were used. For internal staining, cells were fixed at 4×10⁷ cells/ml in 250 μl Cytofix/Cytoperm™ solution (BD Biosciences) for 20 min at 4° C. Washing and staining were then performed in Perm/Wash buffer (BD Biosciences), with 30 min incubation for both primary and secondary antibodies. Internal staining was performed with the following antibodies: Bcl-2 (SC-509), Bcl-xL (SC-8392), Mcl-1 (SC-819) (Santa Cruz Biotechnology, Santa Cruz, Calif.), isotype control antibodies (Sigma), PE-labeled donkey anti-mouse F(ab′)₂ (eBioscience, San Diego, Calif.), and FITC-labeled goat anti-rabbit IgG (ABD Serotec, Raleigh, N.C.). Samples were analyzed by the Guava EasyCyte™ Plus system. Expression was measured as geometric mean fluorescence intensity, subtracting out isotype control staining.

PBMCs were cultured at 10⁶ cells/ml in a 12-well tissue culture plate. Cells were treated for 72 hours with vehicle control or drug for 72 hours, then harvested as above for staining. PBMCs were resuspended at 2×10⁵ cells/tube in 100 μl buffer (HBSS, 0.5% bovine serum albumin, 0.1% sodium azide) and incubated for 30 min with CD3-FITC or CD19-FITC antibodies according to the manufacturer's protocol (BD Biosciences). Cells were then incubated with PE-annexin V and 7-AAD (BD Biosciences) in annexin V binding buffer (BD Biosciences) for 15 minutes prior to analysis of apoptosis on the Guava EasyCyte™ Plus system.

[³H]-thymidine Proliferation Assay

CLL cells (2×10⁵ cells/well) were cultured in quadruplicate in a volume of 200 μl in 96-well tissue culture plates for 96 hours with 1 μM CpG-oligodeoxynucleotides (ODN) DSP30 (Reference 14) (Midland Certified Reagent Company, Midland, Tex.) and 100 units/ml IL-2 (R&D Systems) in the presence or absence of drug. As controls, cells were treated with media without DSP30/IL-2. Cells were incubated with 1 μCi/well [methyl-³H]-thymidine (specific activity 6.7 Ci/mmol, PerkinElmer, Waltham, Mass., USA) for 14-18 hours prior to harvesting onto glass-fiber filters using a semi-automated cell harvester. [³H]-thymidine incorporation was measured as counts per minute (cpm) using a Tri-Carb 2100 timed-resolved liquid scintillation counter (PerkinElmer). The same protocol was utilized for proliferation assays with normal PBMCs, with the exception that growth was stimulated by treatment with 10 μg/ml Phytohemagglutinin-P (PHA, Sigma), 5 μg/ml concanavalin A (ConA, Sigma), 2 μg/ml pokeweed mitogen (PWM, Sigma), 25 ng/ml muromonab-CD3 (α-CD3, Ortho Pharmaceuticals, Raritan, N.J.), or 50 ng/ml phorbol myristate acetate (PMA, Sigma) plus 1 μg/ml ionomycin (iono, Sigma) for 96 hours. Proliferation is reported as percent of control (drug-treated cpm/control-treated cpm×100%).

### Colony Formation Assay

CD34⁺ hematopoietic progenitor cells were isolated from PBMCs from two healthy donors using the CD34 Microbead Kit (Miltenyi Biotec, Auburn, Calif.). 10³ CD34⁺-enriched cells were cultured in a 35 mm culture plate (1000 cells/plate) with or without LMP-420 or fludarabine in 1.1 ml Human Methylcellulose Complete Media (R&D Systems) according to the manufacturer's protocol. Plates were incubated for 14 days at 37° C. in humidified 5% CO₂. Each treatment group was tested in triplicate. Colonies on plates were scored by two independent observers, blinded to the treatment groups, by microscopic evaluation under 10× and 20× power. Colonies were categorized as being of either erythroid or myeloid lineage.

### Potassium Channel Channel Herg Assessment

This assay was performed by MDS Pharma Services. Human recombinant HEK-293 cells over-expressing the human hERG receptor were incubated for 60 min at 25° C. with 1.5 nM [³H] Astemizole in the presence or absence of the indicated concentrations of LMP-420. Nonspecific binding (<10%) was determined by binding of radioligand in 10 μM Astemizole. All samples were determined in duplicate.

### Gene Expression Experiments and Analysis

CLL cells (2.5×10⁷ cells) were cultured in Hybridoma SFM with 10% FBS with or without 2 μM LMP-420 in 100×20 mm tissue culture dishes (BD Falcon). After 24 hours, cells were harvested, washed twice with cold Dulbecco's Phosphate-Buffered Saline (PBS, Sigma), pelleted, and stored at −80° C. Total RNA was extracted from cell pellets using Qiashredder and RNeasy Mini columns (Qiagen, Valencia, Calif.). RNA concentration and quality were assessed using a Nanodrop spectrophotometer (Thermo Scientific) and an Agilent 2100 Bioanalyzer (Agilent Technologies, Santa Clara, Calif.).

RNA samples were prepared and hybridized to U133Plus 2.0 GeneChips (Affymetrix, Santa Clara, Calif.) according to the manufacturer's instructions. All analyses were performed in a minimal information about a microarray experiment (MIAME) compliant fashion. Genomic data are archived in the Gene Expression Omnibus (GEO #GSE20211). Raw data were processed using the Robust Multi-array Average (RMA) algorithm, and underwent subsequent normalization using ComBat to eliminate batch effect (Reference 15). Probe expression from control and LMP-420-treated cells were compared using the Wilcoxon signed-rank test, with a p-value cut-off of less than 0.001 and a Benjamini-Hochberg false discovery rate of <0.05. These analyses were performed using R version 2.7.1.

Cellular pathway induction or repression by LMP-420 treatment was assessed using gene ontology (GO) terms and Kyoto Encyclopedia of Genes and Genomes (KEGG) pathway terms, using Gene Annotation Tool to Help Explain Relationships (GATHER) (Reference 16). Terms with Bayes factors greater than or equal to three were considered significantly enriched. Predictions of genomic signatures of pathway deregulation were evaluated with the binreg algorithm using Matlab (MathWorks, Novi, Mich.) (Reference 17). Pathway signatures for TNF and interferon-alpha (IFN-α) were generated using datasets found at GEO repository numbers GSE2638/2639 and GSE3920, respectively. Binreg parameters used to generate these signatures are listed in Table 4 (below).

### Statistics

Continuous variables were compared using the non-parametric Wilcoxon rank-sum test. Paired Wilcoxon signed-rank testing was performed when appropriate. The Friedman test with Dunn's post-test was used to test for two-way repeated measures analysis among multiple treatment groups. A significance level of less than 0.05 was considered statistically significant.

### Results of Research and Experiments

### CLL Patient Characteristics

Ninety-four consecutively collected CLL samples were used for experiments testing the effect of LMP-420 on CLL cells. The characteristics of the CLL samples used are outlined in Table 1 (below). The majority of samples were obtained from patients with early stage disease who had not required therapy prior to sample collection. However, some samples were obtained from patients with advanced disease and from those with poor prognostic markers such as unmutated IgV_{H} (27%), high CD38 expression (16%), poor risk interphase cytogenetics (11%), and short lymphocyte doubling time (9% of subjects with doubling time of less than 1 year).

### LMP-420 Cytotoxicity for CLL Cells

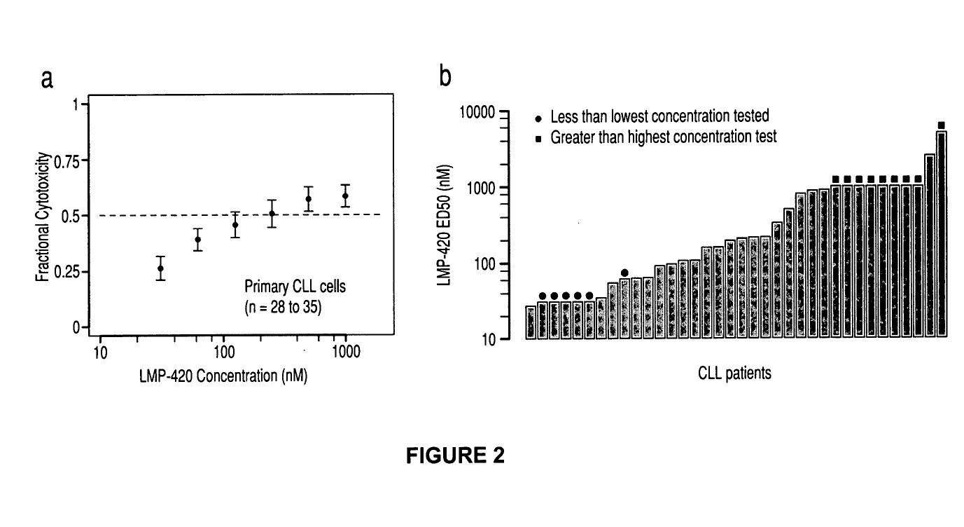

The capacity of LMP-420 to induce cytotoxicity in CLL cells was assessed using the MTS assay. FIGS. 2A-B illustrate cytotoxicity of LMP-420 for CLL cells: (A) Primary CLL cells were isolated freshly from blood of patients and then incubated with serial dilutions of LMP-420, ranging from 30 nM to 1,000 nM (n=28-35 for each concentration) for 72 hours. Cytotoxicity was assessed by MTS assay, and dose response curves were calculated for each patient. Mean fractional cytotoxicity is shown at each concentration. Error bars represent standard error of the mean (SEM), and (B) The dose of LMP-420 inducing 50% cytotoxicity compared to media alone (ED50) is displayed for 36 CLL cell samples. Concentrations tested ranged from 6 nM to 5,000 nM, depending on the sample. Closed circles denote samples for which the ED50 was less than the lowest concentration tested. Closed squares denote samples for which the ED50 was greater than the highest concentration tested. LMP-420 is cytotoxic for CLL cells.

As seen in FIG. 2a, the mean ED50 of LMP-420 for CLL cytotoxicity in 35 samples tested was 245 nM. Calculated ED50 values ranged from less than 30 nM to greater than 5 μM for individual patient samples (FIG. 2b).

### LMP-420 Induction of Time- and Dose-Dependent Apoptosis of CLL Cells.

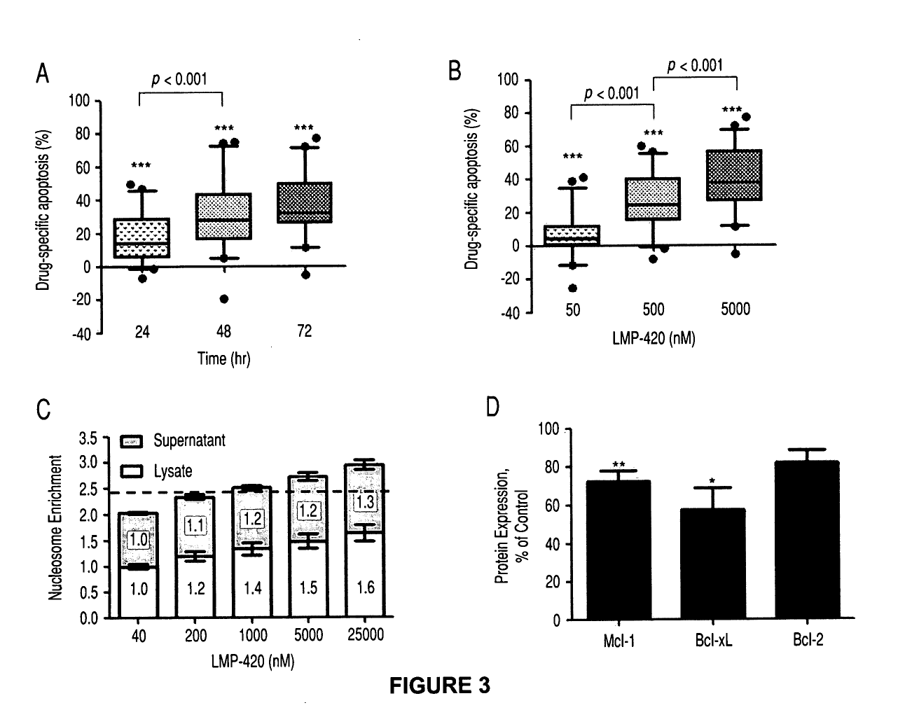

To further characterize the cytotoxic effect of LMP-420 on CLL cells, apoptosis was assessed in a second series of patients. FIGS. 3A-D illustrate LMP-420-mediated apoptosis in CLL cells. Specifically, the charts in (A) and (B) display the 25^{th} to 75^{th} percentiles, with whiskers extending from the 5^{th} to 95^{th} percentiles: (A) Primary CLL cell samples (n=43) were cultured with vehicle control or 5,000 nM LMP-420 for 24, 48, or 72 hours. Drug-specific apoptosis was assessed by annexin V-staining. Box-and-whisker plots demonstrate a statistically significant increase in apoptosis at the three time points (***p<0.0001, Wilcoxon signed-rank test). Time-dependence of the apoptotic effect was also observed (p<0.001, Friedman test with Dunn's post-test), (B) CLL cell samples (n=50) were treated with 50, 500, or 5,000 nM LMP-420 for 72 hours prior to annexin V-staining. Statistically significant apoptosis was observed at each concentration (***p<0.0001, Wilcoxon signed-rank test), and the effect was dose-dependent (Friedman test with Dunn's post-test), (C) CLL patient cells (n=5) were incubated with vehicle control or the indicated doses of LMP-420 for 72 hours. Nucleosome content in the cell culture supernatant and cell lysate was measured by ELISA. The mean with SEM is indicated at each concentration. The dotted line delineates 50% maximal nucleosome enrichment, and (D) Cells from CLL patients were incubated with 500 nM LMP-420 for 72 hours. Expression of Mcl-1 (n=8), Bcl-xL (n=6), and Bcl-2 (n=8) were assessed by flow cytometry, and mean expression was compared to untreated control. Error bars represent SEM. A statistically significant decrease in Mcl-1 and Bcl-xL expression was detected (Wilcoxon signed-rank test, **p<0.01 and *p<0.05, respectively).

In these 43 CLL patient samples incubated with drug for 24, 48, or 72 hours, LMP-420 induced statistically significant apoptosis in a time-dependent manner (p<0.0001, Wilcoxon signed-rank test). Incubation with 5,000 nM LMP-420 resulted in median drug-specific apoptosis of 15%, 28%, and 32% at 24, 48, and 72 hours, respectively (FIG. 3A).

There was a statistically significant dose-dependent increase in apoptosis in a series of 50 CLL samples incubated with 50, 500, or 5,000 nM LMP-420 for 72 hours (FIG. 3B, p<0.0001, Wilcoxon signed-rank test). The highest dose of LMP-420 induced apoptosis in 49 of the 50 samples (98%). Staining indicative of primary necrosis (7-AAD-positive, annexin-V-negative) was negligible in all samples (<2%).

A more pronounced cytotoxic effect was observed by MTS assay than by annexin V staining at 72 hours. To assess the extent to which CLL cells had undergone prior apoptosis and lysis (“secondary necrosis,” Reference 18), we quantified nucleosomes in the cell culture supernatant and in cell lysate at 72 hours (FIG. 3C). Nucleosomes in the lysate indicate intact cells undergoing apoptosis, whereas nucleosomes in the supernatant correspond to cells that have released their contents by secondary necrosis. These latter cells are excluded as debris from flow cytometric analysis of annexin V staining. The dose curve obtained for the cumulative apoptotic effect of LMP-420 is consistent with the average fractional cytotoxicity curve obtained by MTS assay for these five patients (mean ED50 of approximately 850 nM). By contrast, 50% drug-specific apoptosis as assessed by annexin V staining at 72 hours required a mean dose of 5,000 nM in this sample set. Thus, LMP-420 is cytotoxic to CLL by cumulative apoptosis over time.

The apoptotic effect of LMP-420 was associated with decreased levels of the anti-apoptotic proteins Mcl-1, Bcl-xL, and Bcl-2 at 72 hours (FIG. 3D). Cells incubated with 500 nM LMP-420 demonstrated median decreases in Mcl-1 and Bcl-xL expression of 29% (n=8, p<0.01, Wilcoxon signed-rank test) and 41% (n=6, p<0.05), respectively. A trend towards reduced Bcl-2 expression was also observed, with a median decrease of 22% (n=8, p=0.06). The Bax/Bcl-2 ratio was not affected by LMP-420 treatment. LMP-420 reduces expression of Mcl-1, Bcl-xL, and Bcl-2 in CLL cells and induces apoptosis of CLL cells;

LMP-420 Cytotoxicity for CLL Cells from Patients in Poor Prognostic Groups

Drug response was analyzed for CLL samples categorized according to patient characteristics. Cytotoxicity and level of drug-specific apoptosis observed with LMP-420 treatment did not correlate with Rai stage, prior treatment, or cell surface expression of Zap70 or CD38.

However, unmutated IgV_{H} status was associated with significantly greater LMP-420-induced apoptosis than mutated IgV_{H} status (p<0.01, Wilcoxon rank-sum test). The median percent drug-specific apoptosis after 72-hour incubation with 5,000 nM LMP-420 was 34% for the mutated IgV_{H} subgroup (n=30) and 59% for the unmutated IgV_{H} subgroup (n=10). No significant difference in ED50 by IgV_{H} status was observed by MTS assay in a partially overlapping series of patients (p=0.5, Wilcoxon rank-sum test).

Samples from patients with unfavorable cytogenetic profiles were also examined by apoptosis and cytotoxicity assays. 5,000 nM LMP-420 induced apoptosis in patients with 11 q22 deletion or complex cytogenetics (including 11q22 or 17p13 deletion), with ranges from 13-58% (n=3). CLL cell samples with poor risk cytogenetic aberrations had a median MTS ED50 of 332 nM (n=5, range 92 to >1000 nM), while favorable risk cytogenetic aberrations had a median MTS ED50 of 162 nM (n=27, range 27 to 2522 nM) (p=0.3, Wilcoxon rank-sum test).

### LMP-420 Inhibition of CLL Cell Proliferation

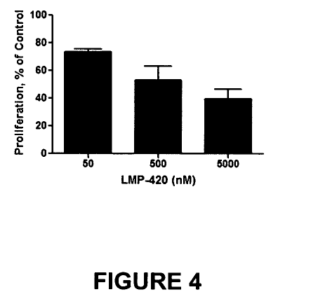

To determine if LMP-420 treatment can inhibit the proliferation of leukemic cells that may be responsible for maintaining the larger population, CLL cells were stimulated to divide in vitro (References 14 and 19). FIG. 4 illustrates inhibition of CLL cell proliferation by LMP-420. CLL cells were incubated with 100 units/ml IL-2 and 1 μM CpG-ODN DSP30 with the indicated concentrations of LMP-420 in quadruplicate for 96 hours. 1 μCi/well [³H]-thymidine was added for the final 14-18 hours of incubation prior to cell harvest onto filters. Thymidine incorporation was measured as cpm. Bars indicate percent mean proliferation relative to untreated control for 3 separate patients. Error bars represent SEM.

LMP-420 inhibits proliferation of CLL cells Incubation with LMP-420 for two hours prior to stimulation suppressed proliferation in a dose-dependent manner (FIG. 4).

### LMP-420 Potentiation of the Cytotoxic Activity of Fludarabine Against CLL Cells

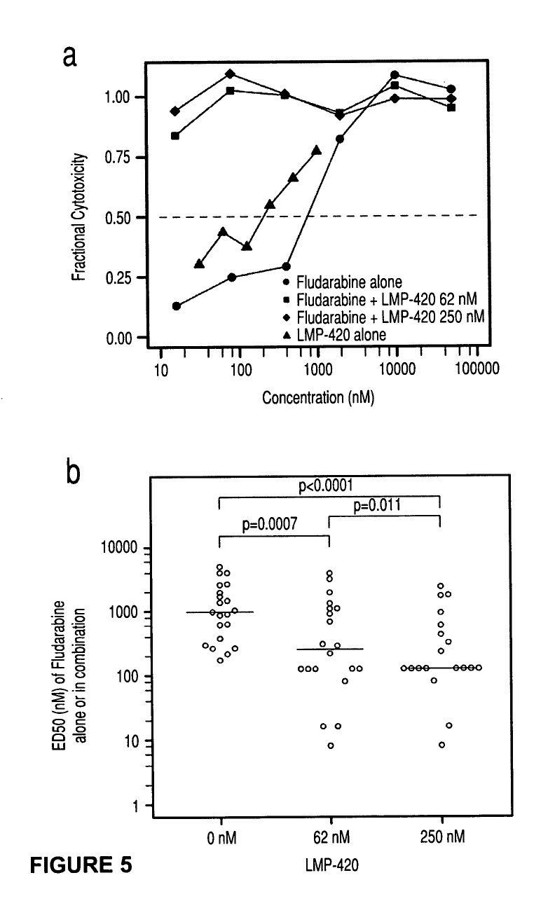

Fludarabine is an integral component of many CLL therapeutic regimens. Consequently, LMP-420 was evaluated to determine if it could potentiate the anti-CLL activity of fludarabine in vitro. FIGS. 5a-b illustrate potentiation of fludarabine cytotoxicity by LMP-420. CLL cells were incubated for 72 hours with serial dilutions of fludarabine alone, serial dilutions of LMP-420 alone, or a combination of serial dilutions of fludarabine with 62 or 250 nM LMP-420. Cytotoxicity was assessed by MTS assay: (A) The dose response curves for a representative patient's CLL cells demonstrate potentiation of fractional cytotoxicity of fludarabine by addition of low concentrations of LMP-420, and (B) Addition of LMP-420 reduced the fludarabine dose required to achieve 50% cytotoxicity. Median ED50 values of fludarabine (denoted by horizontal bars) were 978 nM, 252 nM, and 125 nM when no LMP-420, 62 nM of LMP-420, or 250 nM of LMP-420 was added, respectively. P values were calculated using paired Wilcoxon signed-rank tests. LMP-420 was used at two concentrations, 62 nM and 250 nM. In a representative CLL sample, the addition of low nanomolar concentrations of LMP-420 to fludarabine increased cytotoxicity compared to fludarabine alone (FIG. 5a). In a series of 21 patient samples, both concentrations of LMP-420 resulted in significantly lower fludarabine concentrations required to achieve 50% cytotoxicity (FIG. 5b, p=0.0007 and p<0.0001 for 62 nM and 250 nM, respectively, Wilcoxon signed-rank test). LMP-420 enhances the cytotoxicity of fludarabine for CLL cells;

### Modulation of Immune and NF-Kappa B Pathways by LMP-420 in CLL Cells

We initially hypothesized that LMP-420 induced cytotoxicity and suppressed proliferation of CLL cells by inhibiting TNF production and its subsequent autocrine effects. To determine if this was the case, CLL cells were incubated with serial dilutions of LMP-420 in combination with 1 ng/mL or 25 ng/mL recombinant human TNF. Addition of exogenous TNF did not abrogate the cytotoxicity of LMP-420 at 72 hours (n=9, data not shown). These results were confirmed with apoptosis assays treating cells with 50 ng/mL TNF in combination with LMP-420 (n=4, data not shown). Thus, the mechanism by which LMP-420 is toxic to CLL cells is not likely due to the suppression of TNF production.

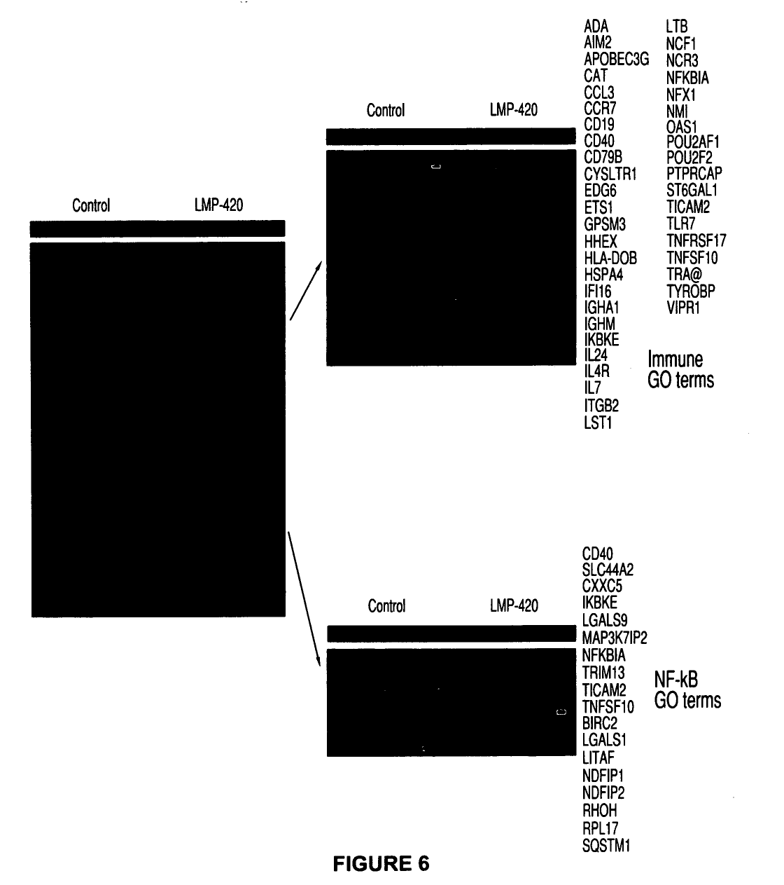

To study the mechanism of action of LMP-420 further, gene expression profiling was performed on CLL cells from thirteen patients after a 24-hour incubation with LMP-420 or media alone. FIG. 6 illustrates gene expression profiling of CLL cells treated with LMP-420. RNA from CLL cells treated in vitro with 2 μM LMP-420 or media alone (“control”) were assayed using gene expression profiling. A total of 1396 differentially expressed gene probes were identified (left heatmap; red color represents up-regulated genes and green color represents down-regulated genes). Immune and NF-kappa B pathway GO terms were significantly represented by these differentially expressed genes (upper right and lower right heatmaps, respectively). Differentially expressed gene symbols in these two pathway groups are listed to the right of the heatmaps. LMP-420 changes expression of CLL cell genes related to immune and NF-kappa B pathways.

Expression of 763 gene probes was increased, and expression of 633 gene probes was suppressed by treatment with 2 μM LMP-420 (FIG. 6). There was a trend toward decreased median expression of TNF in the LMP-420-treated cells compared to the control cells (p=0.08, Wilcoxon signed-rank test). Among the down-regulated genes with LMP-420 treatment, significant enrichment occurred in the GO terms “immune response,” “defense response,” and “response to biotic stimulus” (“Immune GO terms,” FIG. 6), as well as suppression of genes involved in the GO terms “positive regulation of the I-kappa B kinase/NF-kappa B cascade” and “regulation of I-kappa B kinase/NF-kappa B cascade” (“NF-kB GO terms”, FIG. 6). Further characterization of the affected genes in the immune GO terms group using KEGG pathway analysis revealed significant representation of the Toll-like receptor signaling pathway and cytokine-cytokine receptor interactions. Among the genes up-regulated after treatment with LMP-420, there was significant enrichment in the GO terms relating to metabolism (“primary metabolism,” “macromolecule metabolism,” “cellular macromolecule metabolism”, and “macromolecule biosynthesis”) and the NF-kappa B pathway (“regulation of I-kappa B kinase/NF-kappa B cascade”).

Gene expression effects were further assessed by applying genomic signatures of TNF and IFN-α pathway deregulation to the gene expression data. We found a significant reduction in the TNF and IFN-α pathway signature predictions for the LMP-420-treated cells compared to control cells (p=0.027 and 0.003, respectively, Wilcoxon signed-rank test). These genomic alterations in cytokine pathways confirm the results obtained by analyzing gene annotations.

As seen in the gene lists in FIG. 6, RNA expression of CD40 decreased after a 24-hour incubation with LMP-420. We confirmed this result by measuring CD40 surface protein expression after treatment with LMP-420. Consistent with the gene expression results, CD40 protein expression on CLL cells from eight patients decreased by 12.4% at 24 and 33.7% at 72 hours after treatment with 500 nM LMP-420 (p=0.008 for both time points, Wilcoxon signed-rank test).

### Relative Lack of LMP-420 Cytotoxicity for Normal PBMCs and Hematopoietic Precursor Cells

To determine whether the cytotoxic effect of LMP-420 is specific to leukemia cells, PBMCs from healthy donors were incubated with a range of doses of LMP-420 for 72 hours. The median ED50 of LMP-420 on normal PBMCs was not reached at concentrations of up to 512 μM in the MTS assay. The therapeutic index (PBMC ED50/CLL ED50) is therefore greater than 2000, based on the mean ED50 of 245 nM observed in CLL samples in this study. For comparison, the toxicity of several chemotherapeutics currently used in the treatment of CLL was examined, using drug concentrations reported in the literature and observed in MTS assays performed in our laboratory. Using the lowest reported ED50 values for CLL cells, the therapeutic index for each of these drugs is below 30 (Table 2).

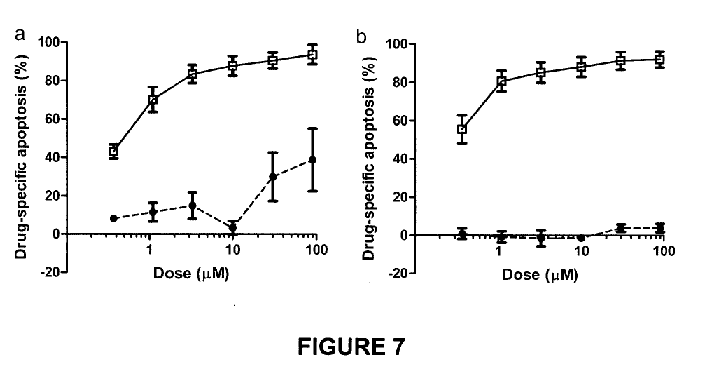

To assess the apoptotic effect of LMP-420 on normal B- and T-lymphocyte subsets, normal PBMCs incubated with LMP-420 for 72 hours were stained with annexin V and CD19 or CD3. FIGS. 7a-b illustrate apoptotic effects of LMP-420 and fludarabine on normal B- and T-lymphocytes. PBMCs were isolated from healthy donors (n=3) by Ficoll-Hypaque gradient density centrifugation. Cells were incubated with vehicle control or the indicated concentrations of fludarabine or LMP-420 for 72 hours. PBMCs were stained with FITC-α-CD19 or FITC-α-CD3, followed by incubation with annexin V and 7-AAD. Cells were analyzed by flow cytometry, and the percent annexin V-positive cells was quantified for the (A) CD19-positive B-cell and (B) CD3-positive T-cell populations. Mean drug-specific apoptosis with SEM is depicted for fludarabine)(□) and LMP-420 ().

As noted in FIG. 7, LMP-420 did not induce significant apoptosis in normal lymphocytes at concentrations up to 90 μM (p>0.05, FIGS. 7a and 7b). By contrast, treatment with 0.4 μM fludarabine caused significant apoptosis in both B- and T-cells (p<0.01 and p<0.05, respectively, Wilcoxon signed-rank test). The average percent drug-specific apoptosis observed after incubation with 1 μM fludarabine—a concentration within its therapeutic range for CLL cells—was 70% for B-cells and 81% for T-cells. By contrast, apoptosis associated with 1 μM LMP-420 was 11% for B-lymphocytes and −1% for T-lymphocytes. Thus, LMP-420 is not toxic to normal B- and T-cells at doses both within and well above its therapeutic dose range.

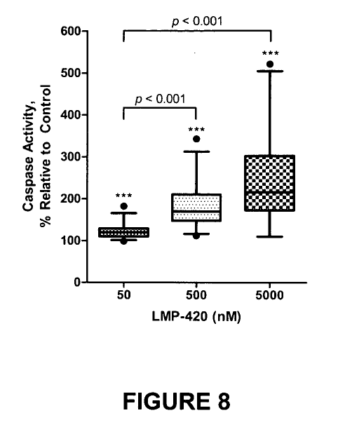

In contrast to normal B and T cells, FIG. 8 illustrates LMP-420 induction of caspase activation in CLL cells. The time- and dose-dependent apoptotic effect of LMP-420 was confirmed by measurement of caspase activity (FIG. 8, n=30, p<0.0001). Primary CLL cell samples (n=30) were cultured with vehicle control, 50, 500, or 5,000 nM LMP-420 for 48 hours. Cells were lysed and caspases 3 and 7 activity were measured by a luminescence-based assay. Caspase activity is expressed as percent luminescence relative to vehicle control. The box displays the 25^{th} to 75^{th} percentiles, with whiskers extending from the 5^{th} to 95^{th} percentiles. Statistically significant caspase activation was observed at each concentration (***p<0.0001, Wilcoxon signed-rank test), and the effect was dose-dependent (Friedman test with Dunn's post-test).

Currently-accepted CLL chemotherapeutics are associated with immuno- and myelosuppression, placing patients at risk for infectious complications during treatment (References 2 and 20). To mimic the proliferative response of immune cells to foreign agents, normal PBMCs from healthy donors (n=3-5) were stimulated for 96 hours with B- and T-cell mitogens in the presence of LMP-420 or other drugs. The doses required to inhibit proliferation by 50% (IC50) in stimulated PBMCs ranged from less than 1 μM to greater than 90 μM (Table 3). The IC50 values for LMP-420 were 100- to 250-fold higher than the mean ED50 of LMP-420 in CLL cells. In contrast, the IC50 values for the other chemotherapeutics tested were no greater than 15-fold above the concentrations of these agents that caused in vitro cytotoxicity to CLL cells (refer to Table 2 for CLL ED50 values). Thus, LMP-420 has dramatically less effect on the proliferation of normal PBMCs at therapeutic CLL cell doses than these cytotoxic chemotherapy agents.

Colony formation assays were performed to examine the effect of LMP-420 on colony formation by hematopoietic progenitor cells isolated from healthy donors (n=2). Erythroid and myeloid colony counts were compared among plates cultured in the presence or absence of various doses of LMP-420 or fludarabine. The mean IC50s of LMP-420 for normal erythroid and myeloid colony formation were 10 and 25 μM, respectively, while those for fludarabine were 5 and 1 μM, respectively. Thus, the IC50s for LMP-420 suppression of both colony types were greater than 40-fold above than the mean cytotoxic ED50 against CLL cells. By contrast, the mean IC50s of fludarabine for suppressing erythroid and myeloid colony formation occurred at concentrations four-fold above and equal to the CLL ED50 for fludarabine, respectively. These results demonstrate the relative specificity in toxicity of LMP-420 for CLL cells compared to normal hematopoietic cells.

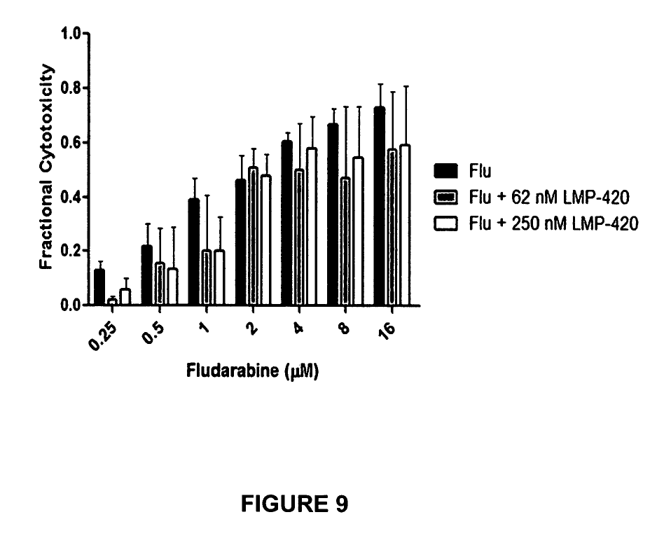

FIG. 9 illustrates lack of LMP-420 potentiation of fludarabine toxicity on normal PBMCs. Although LMP-420 potentiated the cytotoxicity of fludarabine on CLL cells, it did not potentiate the cytotoxicity of fludarabine on normal PBMCs (FIG. 9). PBMCs from healthy donors (n=3) were cultured with vehicle control or serial dilutions of fludarabine (Flu) alone or in combination with 62 or 250 nM LMP-420. After 72 hours, cytotoxicity was assessed by MTS assay. Mean fractional cytotoxicity is shown at each concentration. Error bars represent standard error of the mean (SEM). No significant difference was observed between fludarabine alone or in combination with either dose of LMP-420 (p=0.09, Friedman test with Dunn's post-test).

Lack of Effect of LMP-420 on the Human Potassium Channel hERG.

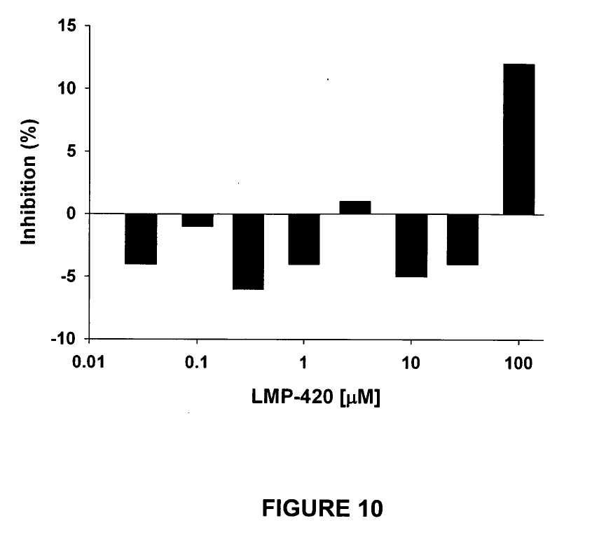

FIG. 10 LMP-420 illustrates that LMP-420 does not bind to the human hERG (human Ether-á-go-go-Related-Gene) cardiac ion channel. This is a key ion channel that has often been affected by drug candidates. Binding of a drug to this channel gives a good prediction of drug-induced cardiac problems and generally eliminates or restricts clinical usefulness of that drug. Our data indicates that the IC₅₀ of LMP-420 for inhibition of radioligand binding to the cardiac potassium channel is >100 μM. LMP-420 does not bind to the human hERG cardiac ion channel.

### DISCUSSION

We have demonstrated that LMP-420 has potent cytotoxic and anti-proliferative effects on primary CLL cells in vitro. Incubation with LMP-420 resulted in time- and dose-dependent loss of viability through primarily an apoptotic mechanism. Normal PBMCs were not significantly affected by LMP-420 at concentrations well above the mean ED50 for CLL cells. LMP-420 had neither the immuno- nor the myelosuppressive properties of fludarabine, but still potentiated the cytotoxic effects of this agent. LMP-420 does not bind to the human hERG (human Ether-α-go-go-Related-Gene) cardiac ion channel. This makes it unlikely that the agent will cause significant cardiac problems in humans. Our data support efficacious and therapeutic uses of LMP-420 for CLL, either alone or in combination with other agents. Combination chemo-immunotherapy with the fludarabine, cyclophosphamide, and rituximab (FCR) regimen is currently the front-line standard of care (References 4 and 21). Co-morbid conditions, advanced age, and infectious complications reduce the number of patients eligible for this treatment and the number of cycles that patients are able to receive (References 2 and 4). Given these barriers to treatment and the lack of full effectiveness of current therapies, better treatments with improved toxicity profiles are needed.

At least one major advantage of LMP-420 over conventional chemotherapy is its specificity for CLL cells over normal cells. Our results demonstrate that LMP-420 induces cytotoxicity and inhibits proliferation in CLL cells but not in normal PBMCs. By contrast, fludarabine concentrations that are toxic to CLL cells also cause greater than 50% apoptosis of normal lymphocytes and suppress mitogen-stimulated proliferation of normal PBMCs. Our prior studies in rodents showed that LMP-420 is non-toxic in vivo at doses achieving a peak plasma level greater than 10 μM. These results suggest that LMP-420 will be a good treatment for CLL therapeutic with low toxicity relative to currently-approved chemotherapies for CLL.

It is important to note that LMP-420 is cytotoxic to malignant cells from CLL patients with a broad range of clinical characteristics. Regardless of Rai stage, ZAP-70 or CD38 positivity, IgV_{H} mutation status, or previous treatment, CLL cells are sensitive to the apoptotic effect of LMP-420. Likewise, apoptosis induced by 5,000 nM LMP-420 was 13 to 58% in samples with unfavorable cytogenetics. There was no significant difference in cytotoxicity between samples with poor-risk (n=5) or favorable-risk (n=27) cytogenetics (ED50 332 vs. 162 nM, p=0.3). Although our analysis of certain subgroups is limited by sample size, we believe that LMP-420 will be useful for the treatment of poor-risk CLL patients for whom current therapeutic options are limited.

LMP-420 induces CLL cell-specific cytotoxicity via apoptotic mechanisms. This may be linked to its ability to alter the cytokine milieu and modulate expression of cell surface receptors involved in interactions with supporting cells in the microenvironment. LMP-420 potently inhibits macrophage and lymphocyte production of TNF and interferon-gamma (Reference 8—unpublished data). Both cytokines can inhibit CLL cell apoptosis, and TNF can also stimulate CLL cell proliferation (References 6 and 22-24). LMP-420 decreases endothelial cell expression of CD40 (Reference 25), a factor implicated in CLL cell survival and proliferation through interactions with CD40 ligand (CD40L) in the microenvironment (References 26 and 27). Likewise, we found significantly decreased cell surface expression of CD40 in CLL cells after treatment with LMP-420. CD40 down-regulation could impact cell survival in our culture conditions by decreasing interactions with CD40L-expressing CLL cells (Reference 28) or the small percentage of T cells potentially remaining after purification.

CLL is thought to be a malignancy with a primary defect in apoptosis, with elevations of Mcl-1 and Bcl-2 contributing to apoptosis resistance (References 29 and 30). Prior research has shown that siRNA suppression of Mcl-1 expression is sufficient to induce apoptosis in CLL cells (Reference 31). We observed decreased expression of the key anti-apoptotic proteins Mcl-1, Bcl-xL, and Bcl-2 in CLL cells after treatment with LMP-420.

Recent studies have indicated that CLL progression involves not only decreased apoptosis, but also in vivo proliferation of a small, but significant subset of the CLL clone (Reference 32 and 33). Higher CLL cell birth rates in vivo are associated with more aggressive disease. Therefore, targeting these proliferating CLL cells has therapeutic importance (Reference 20 and 34). We assessed the effect of LMP-420 on CLL cells that were stimulated in vitro to proliferate with the CpG-ODN DSP30 and IL-2 (Reference 14 and 35). We believe that this stimulated proliferation may reflect the in vivo proliferative compartment, because both higher proportions of proliferating CLL cells in vivo and proliferative response to DSP30 in vitro are associated with progressive disease (Reference 36). LMP-420 significantly inhibited proliferation of in vitro-stimulated patient samples. This demonstrates that LMP-420 has deleterious effects on both resting and proliferating CLL cells. Inhibition of CLL cell proliferation by LMP-420 occurred at concentrations well below those affecting normal PBMC proliferation in response to mitogens or alloantigens. Given this novel specificity for CLL lymphocytes, LMP-420 will be an important adjunct to currently available CLL treatments. It is recommended that LMP-420 be administered intravenously, orally, or subcutaneously at a dosage of 0.1 to 50 mg/kg body weight, when treating a CLL patient.

We initially hypothesized that LMP-420 would be toxic to CLL cells via inhibition of autocrine TNF production. However, our results indicate that the mechanism of action of LMP-420 is more complex, with gene expression profiling suggesting it involves the inhibition of NF-kappa B, toll-like receptor signaling pathways, and cytokine-cytokine receptor interactions. For example, LMP-420 induced the expression of RhoH and SQSTM1, both known to suppress the NF-kappa B pathway (References 37 and 38). Likewise, LMP-420 suppressed the expression of CD40, TICAM2 (TRAM), and TNFSF10 (TRAIL), all of which activate the NF-kappa B pathway (References 39-41). These results provide more explanation for the CLL cell cytotoxic activities of LMP-420, given that constitutive and inducible NE-KB activity support CLL cell survival, and inhibitors of this pathway enhance CLL cell death (References 42 and 43).

One would observe from the above that the present invention demonstrates that LMP-420 is cytotoxic to primary CLL cells from patients with low- or high-risk prognostic factors. Furthermore, the drug potentiates the activity of fludarabine and exhibits a high degree of selectivity for leukemic B cells as compared to normal blood cells. LMP-420 also has the practical benefits of being inexpensive to synthesize, highly stable, well-tolerated in vivo, and orally bioavailable (Reference 44). In addition, LMP-420 has a favorable toxicity profile and efficacy both as a single agent and in combination with fludarabine.

TABLE 1
CLL Patient and Sample Characteristics

| | |
|---|---|
| Total number of CLL samples | 94 |
| Gender (%) | |
| Male | 70 (74) |
| Female | 24 (26) |
| Race (%) | |
| Caucasian | 81 (86) |
| African American | 8 (9) |
| NA | 5 (5) |
| Median age at CLL diagnosis in years (range) | 61 (33-84) |
| Rai stage at diagnosis (%) | |
| 0 | 61 (65) |
| 1 | 18 (19) |
| 2 | 7 (8) |
| 3 | 0 (0) |
| 4 | 4 (4) |
| NA | 4 (4) |
| Rai stage at sample collection date (%) | |
| 0 | 41 (44) |
| 1 | 17 (18) |
| 2 | 12 (13) |
| 3 | 4 (4) |
| 4 | 15 (16) |
| NA | 5 (5) |
| CLL Treatment administered prior to sample | |
| collection (%) | |
| Yes | 29 (31) |
| No | 65 (69) |
| Treatment agent administered (%) | |
| Chlorambucil | 18 (62) |
| Rituximab (single agent) | 7 (24) |
| Purine analogue-based therapy | 15 (52) |
| (fludarabine or pentostatin) | |
| R-CHOP | 1 (3) |
| Bendamustine | 1 (3) |
| IgV_{H} mutation status (%) | |
| Mutated | 64 (68) |
| Unmutated | 25 (27) |
| NA | 5 (5) |
| CD38 status (%) | |
| Positive | 15 (16) |
| Negative | 79 (84) |
| ZAP70 status (%) | |
| Positive | 63 (67) |
| Negative | 31 (33) |
| Interphase Cytogenetics (%) | |
| Normal | 10 (11) |
| 13q14 deletion | 43 (46) |
| Trisomy 12 | 9 (10) |
| 11q22 deletion | 7 (7) |
| 17p13 deletion | 4 (4) |
| NA | 21 (22) |

NA signifies not available.

TABLE 2
Relative ED50 (μM) for CLL cells and PBMCs from normal donors

| | ED50 (μM), | ED50 (μM), | Therapeutic |
|---|---|---|---|
| Drug | CLL* | PBMC | Index |
| LMP-420 | 0.25 | >512 | >2000 |
| Fludarabine | 1.2 | 3.1 | 2.6 |
| Cladribine | 0.01 | 0.1 | 10 |
| Chlorambucil | 3 | 67.7 | 22.6 |
| Cyclophosphamide/ | 5 | 8.5 | 1.7 |
| 4-HC^{†} | | | |
| Bendamustine | 5 | 146 | 29.2 |

*The CLL ED50 value for LMP-420 is the mean value from the MTS experiments described above. The values for other drugs are the minimum values from the range reported in the literature or the mean ED50 observed in MTS assays performed on CLL samples in this study, whichever is lower (References 45-50). The PBMC ED50 values reported are mean values from experiments performed on PBMCs isolated from healthy blood donors (n = 3-6).
^{†}4-hydroperoxycyclophosphamide (4-HC) is a stable precursor to the cyclophosphamide metabolite 4-hydroxycyclophosphamide, which is active in vitro.

TABLE 3
IC50 (μM) for suppression of mitogen-induced proliferation of
normal PBMCs

| | PHA | Con A | PWM | PMA/Iono | α-CD3 |
|---|---|---|---|---|---|
| LMP-420 | >90 | 71 | >90 | >90 | 33 |
| Fludarabine | <3 | <3 | <3 | 0.8 | <3 |
| Cladribine | NT | 0.15 | 0.1 | NT | NT |
| Chlorambucil | 11 | 12 | 7.5 | 7 | 12.5 |
| Bendamustine | 17 | 16 | 14 | 35 | 20 |

IC50 values are based on inhibition relative to vehicle control.
NT indicates not tested.

TABLE 4
Binreg parameters for generating and applying genomic signatures of TNF
and IFN-α pathways

| | TNF signature | IFN-α signature |
|---|---|---|
| N samples, control | 7 | 8 |
| N samples, treated | 7 | 8 |
| Probes | 150 | 100 |
| Factors (metagenes) | 2 | 4 |
| Shift scale normalization | Yes | Yes |
| Quantile normalization | Yes | No |
| Burn-in | 1000 | 1000 |
| Iterations | 5000 | 5000 |

While this invention has been described as having preferred sequences, ranges, steps, materials, structures, features, components, or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention.

The following references, and those cited or discussed herein, are hereby incorporated herein in their entirety by reference.

### REFERENCES

1. Wierda W, O'Brien S, Wen S, et al. Chemoimmunotherapy with fludarabine, cyclophosphamide, and rituximab for relapsed and refractory chronic lymphocytic leukemia. J Clin Oncol. 2005; 23:4070-4078.
2. Keating M J, O'Brien S, Albitar M, et al. Early results of a chemoimmunotherapy regimen of fludarabine, cyclophosphamide, and rituximab as initial therapy for chronic lymphocytic leukemia. J Clin Oncol. 2005; 23:4079-4088.
3. Wadhwa P D, Morrison V A. Infectious complications of chronic lymphocytic leukemia. Semin Oncol. 2006; 33:240-249.
4. Tam CS, O'Brien S, Wierda W, et al. Long-term results of the fludarabine, cyclophosphamide, and rituximab regimen as initial therapy of chronic lymphocytic leukemia. Blood. 2008; 112:975-980.
5. Foa R, Massaia M, Cardona S, et al. Production of tumor necrosis factor-alpha by B-cell chronic lymphocytic leukemia cells: a possible regulatory role of TNF in the progression of the disease. Blood. 1990; 76:393-400.
6. Digel W, Stefanic M, Schoniger W, et al. Tumor necrosis factor induces proliferation of neoplastic B cells from chronic lymphocytic leukemia. Blood. 1989; 73:1242-1246.
7. Ferrajoli A, Keating M J, Manshouri T, et al. The clinical significance of tumor necrosis factor-alpha plasma level in patients having chronic lymphocytic leukemia. Blood. 2002; 100:1215-1219.
8. Haraguchi S, Day N, Kamchaisatian W, et al. LMP-420, a small-molecule inhibitor of TNF-alpha, reduces replication of HIV-1 and Mycobacterium tuberculosis in human cells. AIDS Research and Therapy. 2006; 3:8.
9. Woyach J A, Lin T S, Lucas M S, et al. A phase I/II study of rituximab and etanercept in patients with chronic lymphocytic leukemia and small lymphocytic lymphoma. Leukemia. 2009; 23:912-918.
10. Tsimberidou A M, Thomas D, O'Brien S, et al. Recombinant human soluble tumor necrosis factor (TNF) receptor (p75) fusion protein Enbrel in patients with refractory hematologic malignancies. Cancer Chemother Pharmacol. 2002; 50:237-242.
11. Weinberg J B, Volkheimer A D, Chen Y, et al. Clinical and molecular predictors of disease severity and survival in chronic lymphocytic leukemia. Am J Hematol. 2007; 82:1063-1070.
12. Adams D J, Levesque M C, Weinberg J B, et al. Nitric oxide enhancement of fludarabine cytotoxicity for B-CLL lymphocytes. Leukemia. 2001; 15:1852-1859.
13. Friesen C, Herr I, Krammer P H, Debatin K M. Involvement of the CD95 (APO-1/FAS) receptor/ligand system in drug-induced apoptosis in leukemia cells. Nat. Med. 1996; 2:574-577.
14. Thomas D, Folker S, Manuela K, et al. Immunostimulatory CpG-oligonucleotides induce functional high affinity IL-2 receptors on B-CLL cells: Costimulation with IL-2 results in a highly immunogenic phenotype. Experimental Hematology. 2000; 28:558-568.
15. Johnson W E, Li C, Rabinovic A. Adjusting batch effects in microarray expression data using empirical Bayes methods. Biostatistics. 2007; 8:118-127.
16. Chang J T, Nevins J R. GATHER: a systems approach to interpreting genomic signatures. Bioinformatics. 2006; 22:2926-2933.
17. Bild A H, Yao G, Chang J T, et al. Oncogenic pathway signatures in human cancers as a guide to targeted therapies. Nature. 2006; 439:353-357.
18. Wyllie A H, Kerr J F, Currie A R. Cell death: the significance of apoptosis. Int Rev Cytol. 1980; 68:251-306.
19. Granziero L, Ghia P, Circosta P, et al. Survivin is expressed on CD40 stimulation and interfaces proliferation and apoptosis in B-cell chronic lymphocytic leukemia. Blood. 2001; 97:2777-2783.
20. Morrison V A, Ria K R, Peterson B L, et al. Impact of Therapy With Chlorambucil, Fludarabine, or Fludarabine Plus Chlorambucil on Infections in Patients With Chronic Lymphocytic Leukemia: Intergroup Study Cancer and Leukemia Group B 9011. J Clin Oncol. 2001; 19:3611-3621.
21. Eichhorst B, Hallek M, Dreyling M, Group E G W. Chronic lymphocytic leukemia: ESMO minimum clinical recommendations for diagnosis, treatment and follow-up. Ann Oncol. 2009; 20 Suppl 4:102-104.
22. Reittie J E, Yong K L, Panayiotidis P, Hoffbrand A V. Interleukin-6 inhibits apoptosis and tumour necrosis factor induced proliferation of B-chronic lymphocytic leukaemia. Leuk Lymphoma. 1996; 22:83-90, follow 186, color plate VI.
23. Tangye S G, Raison R L. Human cytokines suppress apoptosis of leukaemic CD5+ B cells and preserve expression of bcl-2. Immunol Cell Biol. 1997; 75:127-135.
24. Buschle M, Campana D, Carding S R, Richard C, Hoffbrand A V, Brenner M K. Interferon gamma inhibits apoptotic cell death in B cell chronic lymphocytic leukemia. J Exp Med. 1993; 177:213-218.
25. Wassmer S C, Cianciolo G J, Combes Vr, Grau G E. Inhibition of Endothelial Activation: A New Way to Treat Cerebral Malaria? PLoS Med. 2005; 2:e245.
26. Grdisa M. Influence of CD40 ligation on survival and apoptosis of B-CLL cells in vitro. Leuk Res. 2003; 27:951-956.
27. Luqman M, Klabunde S, Lin K, et al. The antileukemia activity of a human anti-CD40 antagonist antibody, HCD122, on human chronic lymphocytic leukemia cells. Blood. 2008; 112:711-720.
28. Schattner E J, Mascarenhas J, Reyfman I, et al. Chronic lymphocytic leukemia B cells can express CD40 ligand and demonstrate T-cell type costimulatory capacity. Blood. 1998; 91:2689-2697.
29. Awan F T, Kay N E, Davis M E, et al. Mcl-1 expression predicts progression-free survival in chronic lymphocytic leukemia patients treated with pentostatin, cyclophosphamide, and rituximab. Blood. 2009; 113:535-537.
30. Anurag Saxena S V, Oksana Moshynska, Puneeta Tandon, Koravangattu Sankaran, David P. Sheridan,. Mcl-1 and Bcl-2/Bax ratio are associated with treatment response but not with Rai stage in B-cell chronic lymphocytic leukemia. American Journal of Hematology. 2004; 75:22-33.
31. Longo P G, Laurenti L, Gobessi S, Sica S, Leone G, Efremov D G. The Akt/Mcl-1 pathway plays a prominent role in mediating antiapoptotic signals downstream of the B-cell receptor in chronic lymphocytic leukemia B cells. Blood. 2008; 111:846-855.
32. Messmer B T. In vivo measurements document the dynamic cellular kinetics of chronic lymphocytic leukemia B cells. The Journal of Clinical Investigation. 2005; 115:755-764.
33. van Gent R, Kater A P, Otto S A, et al. In vivo dynamics of stable chronic lymphocytic leukemia inversely correlate with somatic hypermutation levels and suggest no major leukemic turnover in bone marrow. Cancer Res. 2008; 68:10137-10144.
34. Chiorazzi N. Cell proliferation and death: Forgotten features of chronic lymphocytic leukemia B cells. Best Practice & Research Clinical Haematology. 2007; 20:399-413.
35. Decker T, Schneller F, Sparwasser T, et al. Immunostimulatory CpG-oligonucleotides cause proliferation, cytokine production, and an immunogenic phenotype in chronic lymphocytic leukemia B cells. Blood. 2000; 95:999-1006.
36. Longo P G, Laurenti L, Gobessi S, et al. The Akt signaling pathway determines the different proliferative capacity of chronic lymphocytic leukemia B-cells from patients with progressive and stable disease. Leukemia. 2006; 21:110-120.
37. Li X, Bu X, Lu B, Avraham H, Flavell R A, Lim B. The hematopoiesis-specific GTP-binding protein RhoH is GTPase deficient and modulates activities of other Rho GTPases by an inhibitory function. Mol Cell Biol. 2002; 22:1158-1171.
38. Mathew R, Karp C M, Beaudoin B, et al. Autophagy suppresses tumorigenesis through elimination of p62. Cell. 2009; 137:1062-1075.
39. Furman R R, Asgary Z, Mascarenhas J O, Liou2 H-C, Schattner E J. Modulation of NF-{kappa}B Activity and Apoptosis in Chronic Lymphocytic Leukemia B Cells. J. Immunol. 2000; 164:2200-2206.
40. Fitzgerald K A, Rowe D C, Barnes B J, et al. LPS-TLR4 signaling to IRF-3/7 and NF-kappaB involves the toll adapters TRAM and TRIF. J Exp Med. 2003; 198:1043-1055.
41. Jeremias I, Kupatt C, Baumann B, Herr I, Wirth T, Debatin K M. Inhibition of nuclear factor kappaB activation attenuates apoptosis resistance in lymphoid cells. Blood. 1998; 91:4624-4631.
42. Pepper C, Hewamana S, Brennan P, Fegan C. NF-kappaB as a prognostic marker and therapeutic target in chronic lymphocytic leukemia. Future Oncol. 2009; 5:1027-1037.
43. Pickering B M, de Mel S, Lee M, et al. Pharmacological inhibitors of NF-kappaB accelerate apoptosis in chronic lymphocytic leukaemia cells. Oncogene. 2007; 26:1166-1177.
44. Hale L, Cianciolo G. Treatment of experimental colitis in mice with LMP-420, an inhibitor of TNF transcription. Journal of Inflammation. 2008; 5:4.
45. Van Den Neste E, Bontemps F, Delacauw A, et al. Potentiation of antitumor effects of cyclophosphamide derivatives in B-chronic lymphocytic leukemia cells by 2-chloro-2′-deoxyadenosine. Leukemia (08876924). Vol. 13: Nature Publishing Group; 1999:918.
46. Schwanen C, Hecker T, Hubinger G, et al. In vitro evaluation of bendamustine induced apoptosis in B-chronic lymphocytic leukemia. Leukemia. 2002; 16:2096-2105.
47. Silber R, Degar B, Costin D, et al. Chemosensitivity of lymphocytes from patients with B-cell chronic lymphocytic leukemia to chlorambucil, fludarabine, and camptothecin analogs. Blood. 1994; 84:3440-3446.
48. Morabito F, Stelitano C, Callea I, et al. In vitro sensitivity of chronic lymphocytic leukemia B-cells to fludarabine, 2-chlorodeoxyadenosine and chlorambucil: correlation with clinico-hematological and immunopheno-typic features. Haematologica. 1996; 81:224-231.
49. Johnston J B, Paul J T, Neufeld N J, et al. Role of myeloid cell factor-1 (Mcl-1) in chronic lymphocytic leukemia. Leuk Lymphoma. 2004; 45:2017-2027.
50. Kitada S, Andersen J, Akar S, et al. Expression of Apoptosis-Regulating Proteins in Chronic Lymphocytic Leukemia: Correlations With In Vitro and In Vivo Chemoresponses. Blood. 1998; 91:3379-3389.

1. A method of inducing apoptosis or cell death in a leukemia cell, comprising:
subjecting a leukemia cell to a purine nucleoside analogue.

2. The method of claim 1, wherein:
the purine nucleoside analogue includes a boronic acid side chain.

3. The method of claim 1, wherein:
the purine nucleoside analogue comprises 2-amino-6-chloro-9-[5(dihydroxyboryl)-pentyl] purine.

4. The method of claim 3, wherein:
the subjecting step comprises incubating the leukemia cell with the purine analogue having a concentration of about 30 nM to about 5 μM.

5. The method of claim 3, wherein:
the leukemia cell comprises a chronic lymphocytic leukemia cell.

6. A method of inducing apoptosis or cell death in a leukemia cell, comprising:
subjecting a leukemia cell to a purine nucleoside analogue and fludarabine.

7. The method of claim 6, wherein:
the purine nucleoside analogue includes a boronic acid side chain.

8. The method of claim 6, wherein:
the purine nucleoside analogue comprises 2-amino-6-chloro-9-[5(dihydroxyboryl)-pentyl] purine.

9. The method of claim 8, wherein:
the subjecting step comprises incubating the leukemia cell with the purine analogue having a concentration of about 30 nM to about 5 μM.

10. The method of claim 8, wherein:
the leukemia cell comprises a chronic lymphocytic leukemia cell.

11. A method of enhancing cytotoxic activity of fludarabine in inducing apoptosis or cell death in a leukemia cell, comprising:
subjecting a leukemia cell to a purine nucleoside analogue and fludarabine.

12. The method of claim 11, wherein:
the purine nucleoside analogue includes a boronic acid side chain.

13. The method of claim 11, wherein:
the purine nucleoside analogue comprises 2-amino-6-chloro-9-[5(dihydroxyboryl)-pentyl] purine.

14. The method of claim 13, wherein:
the subjecting step comprises incubating the leukemia cell with the purine analogue having a concentration of about 30 nM to about 5 μM.

15. The method of claim 13, wherein:
the leukemia cell comprises a chronic lymphocytic leukemia cell.

16. A method of inhibiting proliferation of a leukemia cell, comprising:
subjecting a leukemia cell to a purine nucleoside analogue.

17. The method of claim 16, wherein:
the purine nucleoside analogue includes a boronic acid side chain.

18. The method of claim 16, wherein:
the purine nucleoside analogue comprises 2-amino-6-chloro-9-[5(dihydroxyboryl)-pentyl] purine.

19. The method of claim 18, wherein:
the subjecting step comprises incubating the leukemia cell with the purine analogue having a concentration of about 30 nM to about 5 μM.

20. The method of claim 17, wherein:
the leukemia cell comprises a chronic lymphocytic leukemia cell.

21. The method of claim 20, wherein:
the method is carried out in vitro or in vivo.

22. A method of treating leukemia, comprising:
administering to a subject in need thereof an effective amount of a purine nucleoside analogue.

23. The method of claim 22, wherein:
the purine nucleoside analogue includes a boronic acid side chain.

24. The method of claim 22, wherein:
the purine nucleoside analogue comprises 2-amino-6-chloro-9-[5(dihydroxyboryl)-pentyl] purine.

25. The method of claim 24, wherein:
the purine analogue is administered intravenously, orally, or subcutaneously at a dose of 0.1 to 50 mg/kg.

26. The method of claim 24, wherein:
the cell comprises a chronic lymphocytic leukemia cell.

27. The method of claim 24, wherein:
the leukemia comprises chronic lymphocytic leukemia.
